(19) United States
(12) Patent Application Publication
(10) Pub. No.: US 2021/0330853 A1
(43) Pub. Date: Oct. 28, 2021

(54) AIR PURIFICATION APPARATUS

(71) Applicant: David Mizandari, Tbilisi (GE)

(72) Inventor: David Mizandari, Tbilisi (GE)

(21) Appl. No.: 17/238,594

(22) Filed: Apr. 23, 2021

Related U.S. Application Data
(60) Provisional application No. 63/015,162, filed on Apr. 24, 2020.

(51) Int. Cl. A61L 9/20; B01D 46/00; B01D 46/46; A62B 7/10; A62B 9/00; A62B 9/02; A62B 18/00; A62B 23/02; A62B 18/08

(52) U.S. Cl. CPC A61L 9/20; B01D 46/0028; B01D 46/46; A62B 7/10; A62B 9/00; A62B 9/02; A62B 18/084; A62B 23/02; A62B 18/08; B01D 2279/40; A61L 2209/12; A61L 2209/14; A61L 2209/111; A62B 18/006

(57) ABSTRACT

Disclosed is an air purification apparatus comprising an air inlet and an air outlet; an air passage in fluid communication with the air inlet and the air outlet comprising a filtration section comprising a HEPA filter; and an ultraviolet (UV) light treatment section, wherein air in the filtration section and the UV light treatment section are treated with UV light.

### CROSS REFERENCE TO RELATED APPLICATIONS

The application claims the benefit of U.S. Provisional Patent Application 63/015,162 filed Apr. 24, 2020, which is hereby incorporated by reference.

### BACKGROUND OF THE DISCLOSED SUBJECT MATTER

SARS-CoV-2 is the official name of the virus responsible for the COVID-19 pandemic, which has resulted in thousands of deaths worldwide and unprecedented levels of economic and social disruption. In the United States alone, over 500,000 deaths have been attributed to COVID-19. The virus was previously referred to as the “Wuhan virus”, “Wuhan coronavirus”, “China coronavirus”, “novel coronavirus” and subsequently “2019-nCoV”. The “SARS” part of the name refers to the new coronavirus' genetic link to the virus that caused the 2003 SARS outbreak. The virus is highly contagious and may have a high mortality rate. The pandemic has resulted in severe social distancing, stay at home orders and other measures to mitigate spread of the virus, which have resulted in significant disruptions of social and economic activity worldwide.

People may be infected with the virus for 1 to 14 days before developing symptoms. The most common symptoms of coronavirus disease (COVID-19) are fever, tiredness, and dry cough. Most people (about 80%) recover from the disease without needing special treatment. However, the disease can be serious and even fatal. Older people, and people with other medical conditions (such as asthma, diabetes, or heart disease), may be more vulnerable to becoming severely ill. Respiratory dysfunction caused by the virus can result in extended treatment with ventilators. The long term effects of coronavirus disease are still under investigation.

Because the virus is highly contagious, it is necessary for essential personnel including for example, health care workers, emergency medical technicians, first responders and other workers in health care settings, to use personal protective equipment (PPE) to prevent them from infection from patients they are treating or the environment around such patients. Subjects that are infected and asymptomatic can spread the virus to others, so other workers that are essential to societal function, including workers in transportation, food and service industries may also need PPE to avoid infection. The severity of the pandemic has led to unprecedented demands for PPE, resulting in shortages of items such as high efficiency breathing masks and respirators.

For example, so-called N95 masks are lightweight, inexpensive breathing masks that are considered as single-use, disposable PPE. They were not intended for long-term use (periods of several hours) and are not easily reusable. Other more durable respirators may be more suitable for long term use but they can be cumbersome and may require special training and fitting for individuals to wear.

It is desirable to develop additional air purification devices such as for use in respirators that fulfill the need for PPE capable of providing breathing air essentially free of viral particles for essential workers.

### SUMMARY OF THE DISCLOSED SUBJECT MATTER

Disclosed herein is an air-purification apparatus comprising an air inlet and an air outlet; an air passage in fluid communication with the air inlet and the air outlet comprising a filtration section comprising a HEPA filter wherein the HEPA filter is treated with UV light while air passes through it; and an ultraviolet (UV) light treatment section wherein filtered air from the filtration section is treated with UV light.

Embodiments of the air purification apparatus include the following, alone, or in any combination.

The air-purification apparatus wherein the air inlet comprises a filter cap comprising a plurality of air inlet holes on the lower portion of the rim of the filter cap.

The air-purification apparatus wherein the inner surface of the filter cap comprises polished aluminum to reflect UV light toward the front face of the HEPA filter.

The air-purification apparatus wherein the HEPA filter further comprises a hydrophobic layer.

The air-purification apparatus wherein the inner surface of the air passage in the UV light treatment section is coated with silver ions or polished aluminum.

The air-purification apparatus wherein the air passage in the UV light treatment section comprises a spiral pathway.

The air-purification apparatus wherein the air passage comprises at least two bends comprising angles of at least 90 degrees wherein the UV light treats the air in the portion of the air passage between the at least two bends.

The air-purification apparatus wherein the UV light is UV-C light with wavelengths in a range of 254 nm to 300 nm.

The air-purification apparatus further comprising an exhalation valve.

The air-purification apparatus further comprising a filter to filter and treat exhaled air.

The air-purification apparatus wherein the apparatus further comprises an impeller disposed in the UV filter section to move air through the air passage.

The air-purification apparatus wherein the air outlet comprises a fitting to attach the apparatus to an article of personal protective equipment or a medical treatment device.

The air-purification apparatus wherein the article of personal protective equipment comprises a respirator mask, hood or shield.

The air-purification apparatus wherein the apparatus further comprises a controller configured to autonomously control airflow through the apparatus by adjusting impeller rotational speed.

Also provided is an air purification device, comprising an air inlet and an air outlet; an internal air passage in fluid communication with the air inlet and the air outlet; an impeller in the internal air passage; a sensor to measure rotational speed of the impeller; a computer instantiated controller; and a non-transitory computer readable storage medium comprising a plurality of computer readable instructions embodied thereon which, when executed by the controller, causes the controller to: receive a plurality of measurements from the sensor, the measurements providing information about impeller rotational speed in the air passage during respiration of a user of the air purification device; average the values obtained from a plurality of measurements obtained during user inhalations and average the values obtained from a plurality of measurements obtained during user exhalations; determine the difference between the average rotational speed value obtained during inhalations and the average rotational speed value obtained during exhalations; compare the difference between the average rotational speed value obtained during inhalations and the average rotational speed value obtained during exhalations with an empirically determined dimensionless factor; and provide instructions to an actuator coupled to the impeller to decrease or increase power supplied to the impeller to decrease or increase impeller rotation speed.

Embodiments of the air purification device include the air purification device further comprising a power consumption sensor coupled to the actuator of the impeller, wherein the controller is configured to: receive a plurality of measurements from the power consumption sensor, the measurements providing information about power consumption during respiration of a user of the air purification device; analyze the power consumption information provided by the plurality of measurements; compare impeller rotational speed to power average values using pulse width modulation (PWM); modify the user-dependent respiration use factor to optimize power consumption; and provide instructions to the actuator coupled to the impeller to decrease or increase power supplied to the impeller to decrease or increase impeller rotation speed in discrete segments.

Also provided is a non-transitory computer readable storage medium comprising a plurality of computer readable instructions embodied thereon which, when executed by a controller of an air purification device comprising an air inlet and an air outlet; an internal air passage in fluid communication with the air inlet and the air outlet; an impeller in the internal air passage; and a sensor to measure rotational speed of the impeller; wherein the plurality of computer readable instructions causes the controller to: receive a plurality of measurements from the sensor, the measurements providing information about impeller rotational speed in the air passage during respiration of a user of the air purification device; average the values obtained from a plurality of measurements obtained during user inhalations and average the values obtained from a plurality of measurements obtained during user exhalations; determine the difference between the average rotational speed value obtained during inhalations and the average rotational speed value obtained during exhalations; compare the difference between the average rotational speed value obtained during inhalations and the average rotational speed value obtained during exhalations with an empirically determined dimensionless factor; and provide instructions to an actuator coupled to the impeller to decrease or increase power supplied to the impeller to decrease or increase impeller rotation speed.

Embodiments include the non-transitory computer readable storage medium wherein the air purification device further comprises a power consumption sensor coupled to the actuator of the impeller, wherein the plurality of computer readable instructions further cause the controller to: receive a plurality of measurements from the power consumption sensor, the measurements providing information about power consumption during respiration of a user of the air purification device; analyze the power consumption information provided by the plurality of measurements; compare impeller rotational speed to power average values using pulse width modulation (PWM); modify the user-dependent respiration use factor to optimize power consumption; and provide instructions to the actuator coupled to the impeller to decrease or increase power supplied to the impeller to decrease or increase impeller rotation speed in discrete segments.

Also provided is a method for controlling air flow through an air purification device comprising an air inlet and an air outlet; an air passage in fluid communication with the air inlet and the air outlet; an impeller in the internal air passage; and a sensor to measure rotational speed of the impeller; the method comprising the controller: receiving a plurality of measurements from the sensor, the measurements providing information about impeller rotational speed in the air passage during respiration of a user of the air purification device; averaging the values obtained from a plurality of measurements obtained during user inhalations and average the values obtained from a plurality of measurements obtained during user exhalations; determining the difference between the average rotational speed value obtained during inhalations and the average rotational speed value obtained during exhalations; compare the difference between the average rotational speed value obtained during inhalations and the average rotational speed value obtained during exhalations with an empirically determined dimensionless factor; and providing instructions to an actuator coupled to the impeller to decrease or increase power supplied to the impeller to decrease or increase impeller rotation speed.

Embodiments of the method include those wherein the air purification device further comprises a power consumption sensor coupled to the actuator of the impeller, wherein the method further comprises the controller: receiving a plurality of measurements from the power consumption sensor, the measurements providing information about power consumption during respiration of a user of the air purification device; analyzing the power consumption information provided by the plurality of measurements; comparing impeller rotational speed to power average values using pulse width modulation (PWM); modifying the user-dependent respiration use factor to optimize power consumption; and providing instructions to the actuator coupled to the impeller to decrease or increase power supplied to the impeller to decrease or increase impeller rotation speed in discrete segments.

Any or all of the foregoing aspects and embodiments, and any other embodiments described in this disclosure can be used alone or in any combination.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 shows an exploded view of an exemplary apparatus in accordance with some embodiments of this disclosure.

FIG. 2 shows an illustration of an exemplary apparatus in accordance with some embodiments of this disclosure.

FIG. 3 shows an illustration of an exemplary apparatus on a human user in accordance with some embodiments of this disclosure.

FIG. 4 shows an exploded side view of another exemplary apparatus in accordance with some embodiments of this disclosure.

FIG. 5 shows a side view of the assembled exemplary apparatus of FIG. 4 in accordance with some embodiments of this disclosure.

FIG. 6 shows a front perspective view of the assembled exemplary apparatus of FIG. 4 in accordance with some embodiments of this disclosure.

FIG. 7 shows a side cross-section view of the assembled exemplary apparatus of FIG. 4 in accordance with some embodiments of this disclosure.

FIG. 8 shows a perspective view of the UV treatment section of the exemplary apparatus of FIG. 4 in accordance with some embodiments of this disclosure.

FIG. 9 shows a bottom perspective view of the assembled exemplary apparatus of FIG. 4 in accordance with some embodiments of this disclosure.

FIG. 10 shows a cross-section view of an exhaled air filter of the exemplary apparatus of FIG. 4 in accordance with some embodiments of this disclosure.

FIG. 11 shows a depiction of the exemplary apparatus of FIG. 4 in accordance with some embodiments of this disclosure.

FIG. 12 shows a depiction of the exemplary apparatus of FIG. 4 worn by a user in accordance with some embodiments of this disclosure.

FIG. 13 shows a functional block diagram of the exemplary apparatus of FIG. 4 in accordance with some embodiments of this disclosure.

FIG. 14 shows the architecture of a controller for the exemplary apparatus of FIG. 4 in accordance with some embodiments of this disclosure.

### SUMMARY OF THE DISCLOSED SUBJECT MATTER

Various aspects of the novel systems, apparatuses, and methods disclosed herein are described more fully hereinafter with reference to the accompanying drawings. This disclosure can, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein, one skilled in the art would appreciate that the scope of the disclosure is intended to cover any aspect of the novel systems, apparatuses, and methods disclosed herein, whether implemented independently of, or combined with, any other aspect of the disclosure. For example, an apparatus may be implemented or a method may be practiced using any number of the aspects set forth herein. In addition, the scope of the disclosure is intended to cover such an apparatus or method that is practiced using other structure, functionality, or structure and functionality in addition to or other than the various aspects of the disclosure set forth herein. It should be understood that any aspect disclosed herein may be implemented by one or more elements of a claim.

Although particular aspects are described herein, many variations and permutations of these aspects fall within the scope of the disclosure. Although some benefits and advantages of the preferred aspects are mentioned, the scope of the disclosure is not intended to be limited to particular benefits, uses, and/or objectives. The detailed description and drawings are merely illustrative of the disclosure rather than limiting, the scope of the disclosure being defined by the appended claims and equivalents thereof.

As used herein, HEPA, which stands for High Efficiency Particulate Air, is a designation used to describe filters that are able to trap 99.97 percent of particles that are 0.3 microns in size.

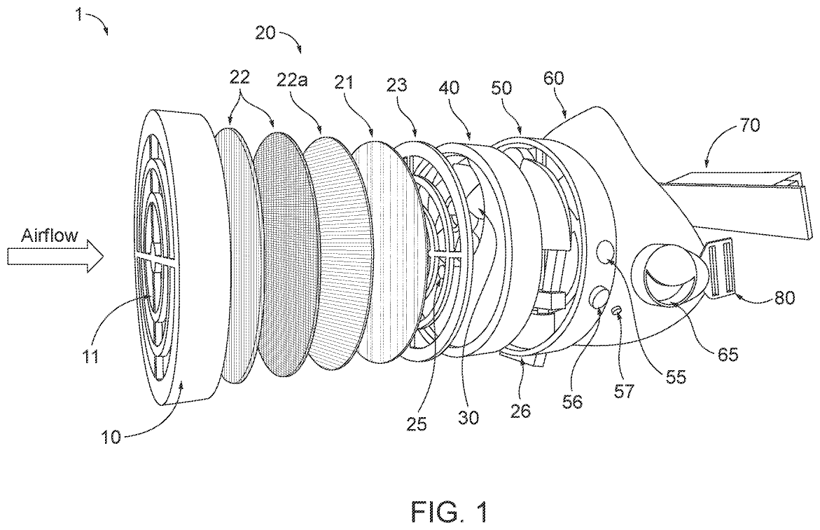

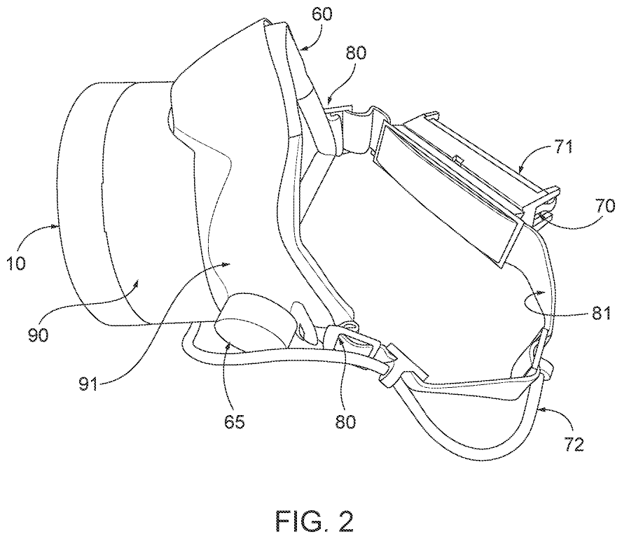

FIG. 1 shows an exploded view of an exemplary apparatus in accordance with some embodiments of this disclosure. In the exploded view of the apparatus shown in FIG. 1, the outer surface of the air passage is not shown to allow depiction of the internal elements of the apparatus. FIG. 2 shows a depiction of an overall view of an exemplary apparatus in accordance with some embodiments of this disclosure, including showing the outer surface of the air passage. The exemplary embodiments shown in FIGS. 1 and 2 comprise an air passage that is generally cylindrical, but this is not limiting. One of ordinary skill can appreciate that the air passage may alternatively comprise cross-sections that are oval, rectangular, square, triangular, tetragonal, or any other shape, or combinations of cross-sections.

As illustrated in FIG. 1, airflow through the apparatus 1 is generally from left to right as shown by the arrow. For simplicity, the airflow through the apparatus is shown generally as linear, but this is not limiting. In other embodiments, airflow may be nonlinear. The air inlet is at the left end of the apparatus as shown in FIG. 1. Air enters the air inlet through a filter cap 10 comprising a plurality of pass-through openings 11 to allow air to enter the filter section 20 of the apparatus. The filter cap 10 is configured to be removably engaged to the filter section to allow access to the filers in the filter section 20 for their insertion, removal, and/or replacement. For example but not limitation, the filter cap may engage the filter section using a friction fit or fasteners such as clips, snaps, tabs, screws, interrupted screws, etc. Optionally, an additional cap without pass-through openings (not shown) may be used to cover the filter cap to protect the air inlet of the apparatus when it is not in use.

Air flows from the air inlet into the filter section 20 of the apparatus comprising a HEPA filter 21, described in more detail below. Optionally, the filter section 20 may comprise one or more pre-filters 22 through which air flows before it reaches the HEPA filter 21. A pre-filter extends the usage life of the more expensive HEPA filter by preventing larger particles from reaching the HEPA filter, thereby reducing fouling of the HEPA filter.

In such embodiments, the first stage in the filtration process comprises one or more pre-filters that remove most of the larger dust, hair, and pollen particles from the air. Pre-filters 22 are also useful to filter out and/or break up larger (e.g. greater than 0.3 microns) aerosol particles in the air or droplets which contain virus particles, such as from exhalations of subjects infected with the virus.

Exemplary embodiments may comprise two types of pre-filters selected from a hydrophobic layer and a fabric mesh layer. The hydrophobic layer is useful for rejecting water drops and moisture. Other pre-filters may comprise, for example, a fibrous or wire mesh that provides a physical barrier to larger particles and aerosols but does not significantly restrict air flow through the apparatus. In the exemplary embodiment shown in FIG. 1, three pre-filters are shown. The first pre-filter may comprise a hydrophobic layer. The next two layers may comprise fabric mesh, the first of which comprises larger mesh openings than the second pre-filter to provide progressively smaller particles passing through the apparatus. In exemplary embodiments, at least one pre-filter comprises a mesh fabric that has 99.9% filtration efficiency for 0.1 micron size particles, and has high fluid resistance of 160 mmHG.

HEPA filters 21 may comprise a mat of randomly arranged fibers. The fibers are typically composed of fiberglass and possess diameters between 0.5 and 2.0 micrometers. Key factors affecting a HEPA filter's functions are fiber diameter, filter thickness, and face velocity. The air space between HEPA filter fibers is typically much greater than 0.3 μm. Unlike membrane filters at this pore size, where particles as wide as the largest opening or distance between fibers cannot pass in between them at all, HEPA filters are designed to target much smaller pollutants and particles. These particles are trapped (they stick to a fiber) through a combination of the diffusion, interception and impaction. To increase the surface area of a HEPA filter for a given cross-sectional area of the air passage, the fibrous mat may be pleated. HEPA filters comprising fiberglass mats are difficult to decontaminate and are typically considered to be disposable after a period of use.

Recently, ceramic HEPA filters have been developed that are more durable than typical fiberglass mat HEPA filters. They may be decontaminated multiple times such as by autoclaving, but are more expensive than fiberglass mat filters.

Preferably, the filter section 20 of the air passage is sized to accommodate commercially available filters, which may be provided in standardized sizes and shapes. In the exemplary embodiment shown in FIG. 1, the filter section comprises a four-stage filter system comprising two pre-filters 22, a charcoal filter 22a and a HEPA filter 21. As shown, each of the pre-filters 22, charcoal filter 22a and HEPA filter 21 are separate filters, allowing for their selective removal and replacement. One or more of the filters may be disposable after a single period of use. Also as shown, the filters are closely spaced and may be in physical contact with each other, but this is not limiting. In alternative embodiments, one or more of the filters may be physically separated from the other filters. For example, a first pre-filter may be integrated with the filter cap so that it may be selectively removed from the filter section and purified by autoclaving. Alternatively, two or more of the filters may be integrated into a single filter module or cartridge. A filter holder 23 is configured to hold the filters in place within the air passage.

To extend the useful lifetime of the HEPA filter while in use, an exemplary embodiment of the apparatus comprises a UV light emitter 25, such as a light emitting diode (LED), that emits UC-C light having wavelengths in a range of about 254 nm to about 300 nm, or from about 260 nm to about 280 nm, or about 260 nm. The HEPA filter 21 is treated with UV light from the LED 25 as air passes through it. For example, the UV light may bathe the face of the HEPA filter facing away from the air inlet, so the UV light passes through the HEPA filter and any pre-filters present in the filter stack. The UV treatment will kill virus particles trapped on the surface of the HEPA filter and pre-filters. One can appreciate that most of the UV light will be blocked from penetrating through all the filter layers and out the air inlet. However, in some embodiments the air inlet may be oriented so that it is not in direct line-of-sight of the UV-emitting diode. It is estimated that about 99% of the virus particles entering the apparatus will be trapped in the filter section of the apparatus. It may take 10 to 60 minutes for an individual virus particle to diffuse through the mesh of the various filters, but most are trapped in the filters. UV light emitted toward the filters may provide about 0.25 to 1.00 mW/sm² (at ambient temperature and humidity) in the filter section, sufficient to kill virus particles trapped at the filter stage. The intensity of the applied UV light may be 30 to 80 times greater than the intensity needed to kill virus on a surface in clinical studies.

Normal breathing rhythms may be sufficient to draw air through the air passage during the inhalation phase of respiration of a user. However, in some instances, such as during physical activity or in subjects with respiratory difficulty, the subject may need assistance drawing air through the apparatus. In exemplary embodiments, the apparatus has a BOOST AIR-ON function, wherein the air purification apparatus further comprises a propeller or impeller 30 powered by a micro-engine (not shown) to facilitate movement of air through the air passage. BOOST power can be controlled by user input to be set at fixed values such as low/medium/high or variably controlled by user input by use of a variable component such as a rheostat. In some embodiments, the augmented air flow may be intermittent, timed to provide air flow augmentation during inhalation and halt air flow augmentation during exhalation, according to the breathing rhythm of the subject. In other embodiments, powered air flow may be continuous.

The apparatus also comprises a UV treatment section 50 for treating the airflow with UV light, preferably UV-C light having wavelengths in a range of about 254 nm to about 300 nm, or from about 260 nm to about 280 nm, or about 260 nm provided by one or more UVC-C light emitters (not shown in FIG. 1). This provides a second treatment area to neutralize any virus particles that may have passed through then filter section. In some embodiments, the inner surface of the air passage in the UV light treatment section 50 is coated with silver ions or polished aluminum. Such coatings may provide a reflective surface to scatter the UV light throughout the UV treatment section to provide good contact with virus particles therein. Further, silver ions are known to provide biocidal effects, so the interior of the apparatus may be protected from mold, fungus and/or bacteria growth while the apparatus is not in active use (i.e. when the UV treatment is activated).

The air passage in the UV treatment section 50 comprises a convoluted pathway or nonlinear pathway. This serves two purposes. First, the convoluted pathway provides an air passage of sufficient volume to provide adequate dwell time of air in the UV treatment section to kill air-borne virus particles. Second, the convoluted pathway serves as a light trap to contain the UV light in the UV treatment section to minimize exposure of the user to the harmful effects of UV light.

In some embodiments, the air passage in the UV light treatment section comprises a spiral pathway.

In some embodiments, the air passage in the UV light treatment section comprises at least two bends comprising angles of at least 90 degrees wherein the UV light treats the air in the portion of the air passage between the at least two bends.

In some embodiments, the air passage in the UV light treatment section comprises UV-C emitting diodes at the at least two bends configured to emit UV light into the portion of the air passage between the at least two bends.

In some embodiments, the air passage in the UV light treatment section comprises a Z-shaped passage.

In some embodiments, the air passage in the UV light treatment section comprises a labyrinthine pathway.

In some embodiments, the pathway may comprise an entrance portion that leads in the same general direction as the overall airflow through the apparatus e.g. the left-to-right direction shown in FIG. 1). Following the entrance portion, the pathway may make a 90 degree turn such that the pathway in the UV-treatment portion travels within a plane perpendicular to the general airflow through the apparatus, with embodiments of the convoluted pathway exemplified above. After the convoluted portion, the pathway may make another 90 degree turn such that the pathway in this exit portion resumes travel in the general direction of air flow indicated. This configuration provides the pathway in a compact, planar configuration suitable for incorporation in a portable and/or wearable device.

In some exemplary embodiments, the material comprising the boundaries of the air passage within the UV treatment section comprises a material opaque to UV light, such that the UV light is contained within the UV treatment section. In these embodiments, the UV light may be emitted from point sources and is directed through the air pathway.

In other exemplary embodiments, the material comprising the boundaries of the air passage passes comprises a material transparent to UV light, and the UV light is emitted across or orthogonal to the air pathway from UV emitters that provide illumination in an area roughly coequal to the area of the pathway. In such embodiments, the UV treatment section is desirably contained within a material opaque to UV light, such that the UV light is contained within the UV treatment section.

The air passage in the UV light treatment section 50 is configured to provide a residence or dwell time of air within the UV light treatment section sufficient to kill virus particles that escape the filter stage and enter the UV light treatment section. Air-borne particles may absorb UV-C light more readily than on a surface, but clinical studies for sterilization of air volumes are limited. The air pathway in this section is configured to move air in close proximity to UV LEDs to maximize the local intensity of light impinging on the virus particles, such as with UV LEDs positioned in corners of the pathway. In other areas of the convoluted pathway, reflected UV light continues to interact with air-borne virus particles.

The UV intensity is selected in a way to completely neutralize pathogens including virus particles such as SARS-CoV-2. If the UV rays are utilized with lesser intensity or a shortened time they will be less effective. The LED spectrum should also be selected at a proper viricidal level to completely destroy the virus particle and neutralize its reproductive abilities. Notably, the UV light is UV-C light with wavelengths in a range of about 254 nm to about 300 nm.

The disclosed apparatus optionally comprises a negative ion generator 40 to treat the air with negative ions, for example, within the convoluted pathway in the UV treatment section or in a subsequent ionization chamber section. As the air passes through the ionization chamber, particles in the air are ionized to negatively charged particles that can be removed from the air stream by electrostatic precipitation. When the apparatus described herein is intended for use in PPE, the negative ion generator may be not be used or is configured to generate negative ions with power levels that do not provide static charge within the device that require grounding or generate ozone. The collector plates can be removed from the apparatus for cleaning.

Following the filter section, UV treatment section and optional ionization sections, the now-purified air, estimated to be about 99.999% clean air, enters the air outlet section. In some embodiments, a one way valve (not shown in FIG. 1) between the air purification section and the air outlet allows passage of pure air out of the purification section while preventing exhaled or contaminated air from entering. The air outlet section may comprise a holding chamber to contain a volume of purified air. For a breathing mask or respirator, described in more detail below, the holding chamber may have a volume equivalent to about one breath of a typical respiratory cycle.

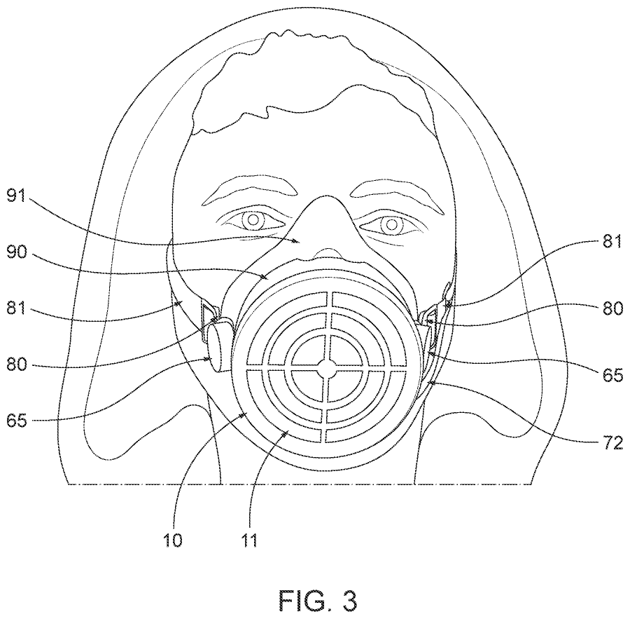

As shown in FIGS. 2 and 3, the apparatus is at least partially contained in an impermeable casing comprising two parts 90 and 91 that provides the outer surface of the air passage such that air can enter or leave the apparatus only through the air inlet and air outlet ends of the air passage. One of ordinary skill in the art can appreciate that the air inlet and air outlet allow air to enter and exit the interior of the apparatus, respectively.

The apparatus may comprise a power supply to provide power for the UV emitting LEDs, the micro-motor to facilitate air movement through the apparatus and other electrical components of the device. The power supply may comprise a power cord to obtain electrical power from an external power source such as an AC power source or a converter to convert AC power to DC power. Power supply may be any supplier of energy, including wall sockets and electronic devices that convert solar, wind, water, nuclear, hydrogen, gasoline, natural gas, fossil fuels, mechanical energy, steam, and/or any power source into electricity. Preferably the power source comprises a battery 71 or 471. In exemplary embodiments, the power supply may include one or more batteries 71, including, without limitation, lithium, lithium ion, nickel-cadmium, nickel-metal hydride, nickel-hydrogen, carbon-zinc, silver-oxide, zinc-carbon, zinc-air, mercury oxide, alkaline, or any other type of battery known in the art. Certain batteries may be rechargeable, such as wirelessly (e.g., by resonant circuit and/or a resonant tank circuit) and/or by plugging into an external power source. In some embodiments the battery may be contained in a battery compartment integrated into the apparatus. In other embodiments, such as shown in FIGS. 1 and 2, the battery 71 may be contained in a battery compartment or case 70 external to the apparatus and in electrical connectivity to the apparatus via a power cord 72. In an exemplary embodiment, the apparatus comprises a battery that will allow 8-12 hours of use with one charge. In this embodiment of the battery, the charge time may be 1-3 hours. In other embodiments the battery working duration is 6-8 hours.

The apparatus may also comprise one or more switches, such as a power on/off switch 55, shown in FIG. 1, to activate or inactivate the apparatus. Other switches (not shown in the Figures) may selectively activate or deactivate one or more individual electrical components of the apparatus such as the BOOST Air micromotor, the negative ion generator 40, one or more of the UV LED emitters, etc. The apparatus may also comprise one or more indicator lights (not shown in the figures) to indicate the on/off status of the apparatus and/or individual electrical components of the apparatus, such as those listed above.

The apparatus may optionally further comprise an external UV light 26 such as a UV LED for illuminating small objects outside the apparatus (e.g. phone, wallet, medical device, door handle, switch, pen, keyboard, touchpad, touchscreen, etc.) to provide surface decontamination of such objects. Preferably the UV wavelength for the external UV light is about 222 nm, which is not harmful for human skin and eyes. The external UV light can be activated by an external button 56 for a brief period, such as 5 to 30 seconds, for example 10 seconds, for sterilizing items. An indicator light may indicate when the UV LED is activated, because UV light is not visible. As shown in FIG. 1, the external UV light and power button may be integrated onto the exterior housing of the apparatus. In other embodiments (not shown), the external LED, power button and/or indicator light may be contained in a small auxiliary handheld device connected to the apparatus or the battery compartment by a power cord to facilitate its use without the user needing to position the entire apparatus to aim the UV light at the object(s) to be sterilized.

The apparatus may optionally comprise one or more sensors to measure or assess conditions related to its operation. For example, the apparatus may comprise an ambient air pressure sensor 57, shown in FIG. 1. Humidity and/or temperature sensors (not shown) may also be included.

In some aspects the apparatus is used to purify breathing air prior to its inhalation to protect an uninfected user. These aspects may include the following embodiments.

The air outlet comprises a fitting to attach the apparatus to an article of personal protective equipment, such as a respirator mask, hood or shield, or a medical treatment device.

The air outlet is integrated into an article of personal protective equipment, such as a respirator mask, hood or shield.

The apparatus wherein the respirator mask, hood or shield further comprises an exhalation valve.

As shown in FIGS. 1 through 3, the apparatus described herein may be integrated into an article of personal protective equipment (PPE). In the embodiments depicted in FIGS. 1 through 3, the PPE may comprise a mask. In the embodiment shown in these Figures, the mask is an oronasal mask configured to cover the nose and mouth of a user. The air outlet of the apparatus provides purified air to the interior of the mask for breathing by the user. The mask may comprise an inner layer comprising an elastomeric, soft, or foam material 60 as shown in FIG. 1 to provide sealing to the face of the user so that all air breathed by the user passes through the disclosed air purification apparatus. The mask may also comprise an outer layer comprising rigid material, shown in FIGS. 2 and 3 wherein the rear portion of the external casing 92 is shaped to cover the nose and mouth of a user, to provide structure and stability to the mask. The inner and outer layers of the mask are preferably shaped to approximate the surface contour of a human user to facilitate its sealing to the user and/or provide a comfortable fit. The facial conformity of the mask portion may advantageously be sized for different facial features. Optionally, a mask portion may be customized to an individual user by measuring the user's facial contours and programming those contours into a 3D printer to fabricate a facial portion of the mask that closely conforms to that individual's facial contours.

The mask also comprises means to hold the mask in place on the user's face. Typically the means comprise one or a plurality of straps 81 (not shown in FIG. 1, shown in FIGS. 2 and 3), bands, harness or the like that engage one or more areas of the user's head, neck, ears, etc. to hold the mask in place. The straps are adjustable and/or elastic to hold the mask snugly against the user's face to facilitate an air seal. The mask also comprises strap holders 80 to attach the strap to the mask. In the embodiment shown in the Figures, a battery compartment 70 engages the strap 81 to hold the battery. Positioning the battery compartment on the strap allows the weight of the battery to be distributed away from the front of the mask, which may provide increased comfort for the user. Other locations for the battery compartment may include in an auxiliary unit to be carried in a pocket, or positioned at the chest, waist, upper or lower back, etc. The mask also comprises an exhalation valve 65 to direct exhaled air out of the mask and not back through the air passage of the purification apparatus.

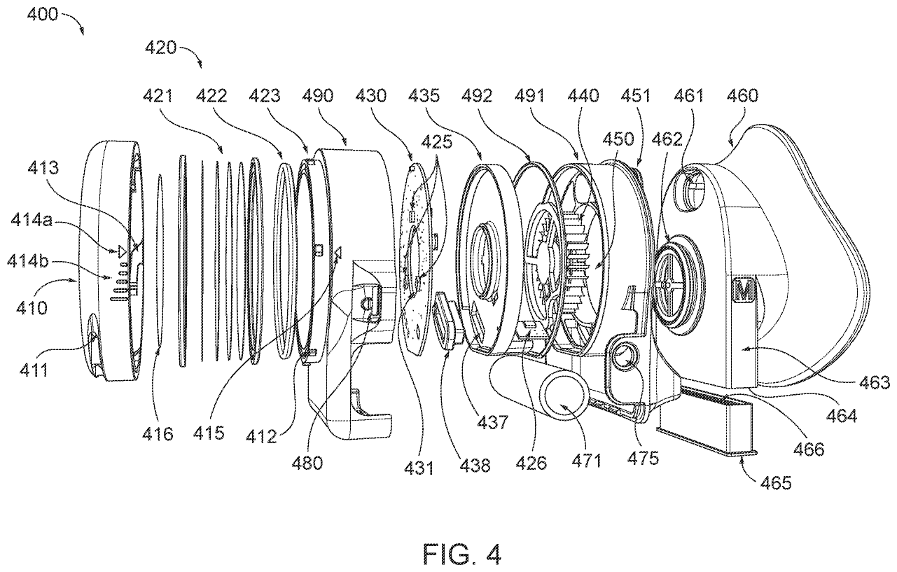

FIG. 4 shows an exploded view of another exemplary apparatus in accordance with some embodiments of this disclosure.

As illustrated in FIG. 4, airflow through the apparatus 400 is generally from left to right. The air inlet is at the left end of the apparatus as shown in FIG. 4. Air enters the air inlet through a filter cap 410 comprising a plurality of pass-through openings 411 to allow air to enter the filter section 420 of the apparatus. The openings 411 are disposed on the lower (as worn by the user) portion of the rim of the filter cap 410. The filter cap 410 is configured to be removably engaged to the filter section to allow access to the filers in the filter section 420 for their insertion, removal, and/or replacement. For example but not limitation, the filter cap may engage the filter section using a friction fit or fasteners such as clips, snaps, tabs, screws, interrupted screws, etc. In the embodiment shown, a plurality of tabs 412 on the filter holder 423 of front housing body 490 are configured to engage complementary slots 413 on the inside of the rim of filter cap 410. Alignment arrows 414a on the outside of the rim of filter cap 410 and the outside of front housing body 490 provide visual indicators to help a user to engage the tabs 412 to the slots 413. Rotating the filter cap 410 locks the tabs 410 into the slots 413 and holds the filter cap 410 on the housing body 490. Raised ribs 415 on the filter cap 410 provide a gripping surface to facilitate a user rotating the filter cap 410. Optionally, an additional cap without pass-through openings (not shown) may be used to cover the filter cap 410 to protect the air inlets 411 of the apparatus when it is not in use. A reflective disk 416 comprising for example polished aluminum is disposed on the inside surface of the filter cap 410 to reflect UV light back through the filter as described below.

The filter section 420 is disposed between the filter cap 410 and on the filter holder 423. A HEPA filter 421 is disposed in the filter section 420. In embodiments, the apparatus 400 does not comprise pre-filters as described above and comprises only HEPA filters that are UV-C transparent and may optionally comprise hydrophobic layers thereon. In the embodiment shown, the HEPA filter 421 comprises two N99 HEPA layers disposed between hydrophobic layers on front and back sides held together by a perimeter ring. An O-ring 422 seals the junction between filter cap 410 and filter holder 423.

A printed circuit board assembly (PCBA) 430 is disposed behind the filter holder 423 and inside front housing body 490. The PCBA includes all circuitry and components for processors, controllers, memory, etc. as described in greater detail below. The PCBA also supports one or more UV-C emitters (LEDs) 425 disposed to illuminate the HEPA filter 421 to irradiate and kill virus particles that are collected on the filter 421. Three UV-C LEDs 425 are shown is this embodiment, but this is not limiting. The rear (as worn by the user) of the filter 421 is sterilized by direct irradiation by UV-C light from emitters 425. A portion of the UV-C light passes through the filter 421 and is reflected back to the filter 421 by reflective disk 416, sterilizing the front (as worn by the user) surface. This provides for significant (up to 100%) of viral and germ particles that enter the device before they can pass through the filter 421.

A central hole 431 in PCBA 430 allows air to pass through the PCBA 430 into the next section of the apparatus. A PCBA holder 435 is disposed inside of front housing body 490 to hold PCBA 430 in place. PCBA holder 435 comprises a central hole 436 coaxial with hole 431. PCBA holder 435 also comprises a second opening 437 to allow a wiring harness (not shown for simplicity) to pass through. The wiring harness comprises connections between the components on the PCBA 435 and the other electric components of the apparatus contained in the rear housing body 491. A connector seal 438 comprising elastomeric material is disposed in the second opening 437 to seal the opening to prevent air flow through opening 437. An O-ring 492 seals the junction between the PCBA holder 435 and the rear housing body 491.

Air from the filter section 420 passes through openings 431 and 436 into the UV treatment chamber 450 disposed in the rear housing body 491. An impeller 440 comprising a plurality of curved vanes disposed in UV treatment chamber 450 is rotated within the chamber 450 by a micro-motor (not shown) to move the air in a spiral movement. Notably, the impeller comprises a material that is transparent to UV-C light to allow it to illuminate air throughout the UV treatment chamber. The speed of rotation may be controlled by a controller disposed on PCBA 430. UV-C LED 426 is disposed proximate the side of the UV treatment chamber 450 to illuminate air and any remaining viral particles in the chamber 450 to provide a second UV treatment. In embodiments, optionally one or more UV-C LEDs may be disposed on the rear of PCBA support 435 to illuminate the UV treatment chamber 450. Following this UV treatment, air passes out of the chamber 450 through air passage 451 disposed at the upper side of the apparatus into opening 461 in the face mask 460. Mask 460 is configured to be releasably attached to the rear housing body 491 so that it can be removed for separate decontamination/sterilization and/or replacement of the mask 460 and/or exhaled air filter cartridge 465. Mask 460 comprises a flexible material such as silicone configured to conform to a user's face and cover the user's nose and mouth and may be configured in different sizes (e.g. small, medium and large) to provide better fit for different users. A one-way exhale valve 462 is disposed in mask 460. The lower portion of mask 460 comprises a chamber 463 with an opening 464 configured to receive a filter cartridge 465 having a filter 466 to filter the exhaled air from the user.

Front housing body 490 and rear housing body 491 are configured to be engaged or attached together to define a compact unitary housing that encloses all the internal components of the apparatus. They may comprise rigid plastic moldings with generally smooth exterior surfaces that can be easily wiped for surface decontamination. For example but not limitation, the front housing body 490 may engage the rear housing body 491 using a friction fit or fasteners such as clips, snaps, tabs, screws, interrupted screws, etc. When engaged, the front housing body 490 and rear housing body 491 define a compartment below the air passage to contain a battery 471 and other electrical components (not shown) such as a wiring harness to connect the PCBA 430 with the battery 471 and power and/or control switches. In embodiments, the housing may comprise a door to access the battery 471 for replacement. In other embodiments, the device may comprise a port configured to connect a power cord to an external power source to recharge a rechargeable battery 471. Openings 475 on each side of rear housing body 491 provide for positioning switches or other user control features (not shown).

Front housing body 490 also comprises strap attachment features 480 on each side to allow for attaching a head strap or harness (see Figure Y) to allow a user to dispose the apparatus 400 against the user's face to cover the user's nose and mouth to breathe through the apparatus while keeping the user's hands free for other tasks.

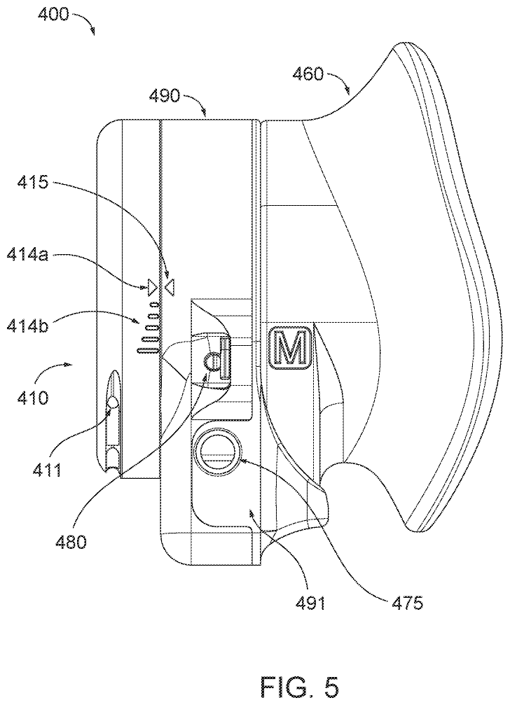

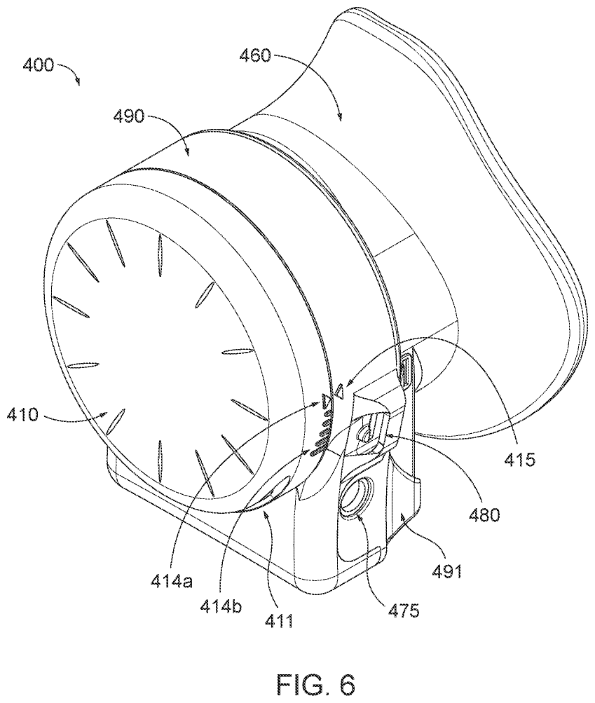

FIGS. 5 and 6 show the assembled apparatus 400 in side and front perspective views.

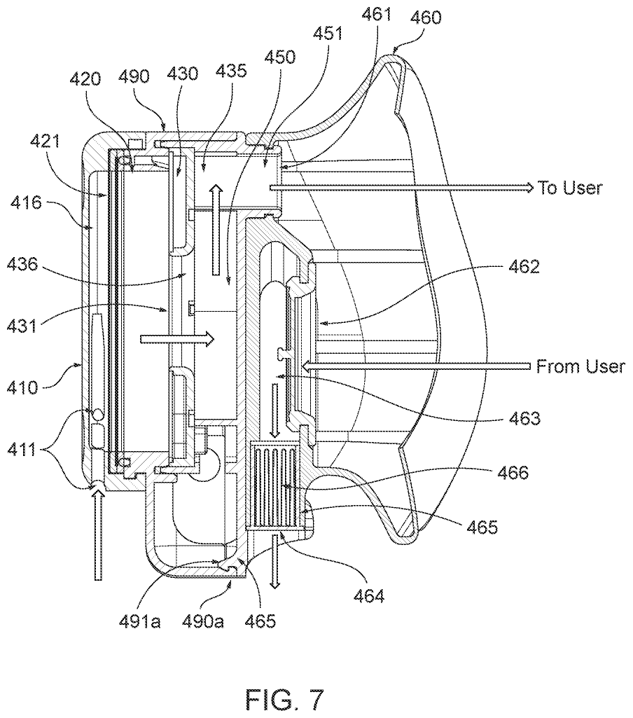

FIG. 7 shows a cross section of the assembled apparatus 400 to show the air flow through the apparatus. Ambient air enters the apparatus 400 through air inlet holes 411 disposed on the lower portion of the filter cap 410, then is drawn through the HEPA filter 421 in the filter section as shown by the open arrows. While in the filter section, air is illuminated with UV-C light from UV-C LEDs 425 disposed on the front face of PCBA 430 as indicated by the dashed arrows. The configuration of the device provides active irradiation of the filter and air passing through it with UVC from the rear, and reflective disk 416 reflects UV-C back toward the front face of the filter, so passing particles in the air are always going through direct and indirect UV-C illumination. The UV-C light also kills viral particles that are trapped on the HEPA filter 421.

Air passes through hole 431 in PCBA 430 and hole 436 in PCBA holder 435 into the UV treatment chamber 450, where it is treated a second time with UV-C light from UV-C LED 426 as indicated by the dashed arrow. For simplicity of illustration, impeller 440 is not shown. After treatment in the UV treatment chamber 450, air passes out of the chamber 450 through air passage 451 disposed at the upper side of the apparatus into opening 461 in the face mask 460.

A user wearing the mask inhales air from the mask 460 and exhales air back into mask 460. Exhaled air passes through one-way valve 462 into chamber 463, through filter 466 in filter cartridge 465 and out opening 464 into the ambient air.

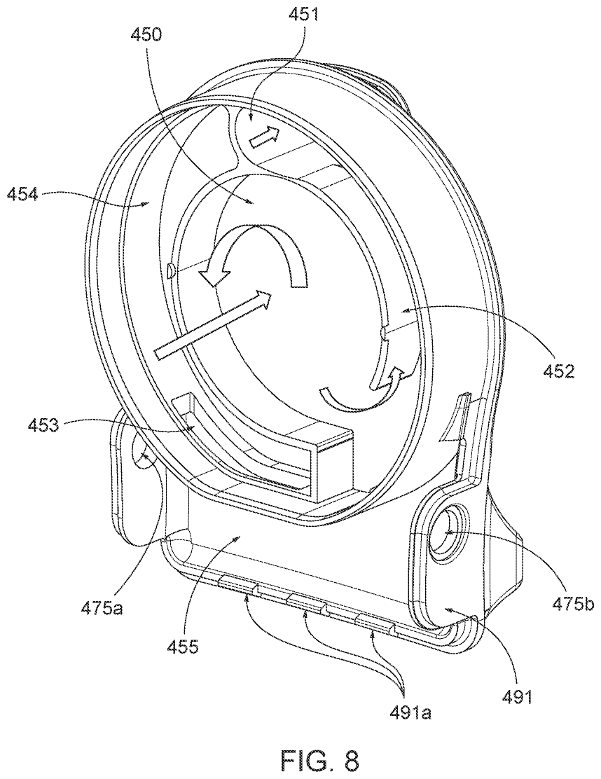

Also shown in FIG. 7 are interlocking tabs and flanges 490a and 491a that hold front housing body 490 and rear housing body 491 together. FIG. 7 shows only one set of tabs and flanges 490a and 491a, but they are a subset of a plurality of interlocking tabs and flanges (see FIG. 8).

FIG. 8 shows a perspective view of rear housing body 491, which comprises UV treatment chamber 450 as a molded-in feature. Air flow is indicated by open arrows. Air enters the UV treatment chamber 450 from hole 436 in PCBA holder 435, not shown in this figure and is rotated around the UV treatment chamber 450 by impeller 440 (not shown) in a spiral fashion. Air then passes out of the chamber 450 into chamber 452 and through air passage 451 disposed at the upper side of the apparatus into opening 461 in the face mask 460. While air is in the UV treatment chamber 450, it is treated with UV-C light from LED 426 as indicated by the dashed arrows. LED 426 has a 120-degree lighting angle, so it will emit UC light into the internal volume of chamber 450 and chamber 452. The inner surfaces of the air passage in chambers 450, 452 and air passage 451 are coated with silver ions or polished aluminum to reflect the UV-C light. Due to the reflecting coating inside the UC treatment section, UV-C light will be reflected and diffused throughout chambers 450, 452 and air passage 451. Optionally, UV-C LEDs may also be disposed on the rear of PCBA holder 435 to shine UV-C light axially into chamber 450.

Also shown in FIG. 8 is slot 453 for cabling to pass through, for example, wiring (not shown for simplicity) to provide electrical connectivity for components such as power connections, an on/off button 476 in hole 475a, an LED for the power button, a charging socket in the hole 475b, battery thermistor, and the like. Chamber 454 is configured to contain larger electrical and electronic parts that do not fit conveniently directly on PCBA 430, such as coils and processors, etc. Chamber 454 provides a physical separation between these components and the impeller 440 so that they do not contact the impeller and impede its rotation. The lower part of rear housing body 491 is configured to combine with the lower part of front housing body 490 (not shown) to define a chamber 455 for holding battery 471 and mechanical components for power (on/off) and other switches or controllers.

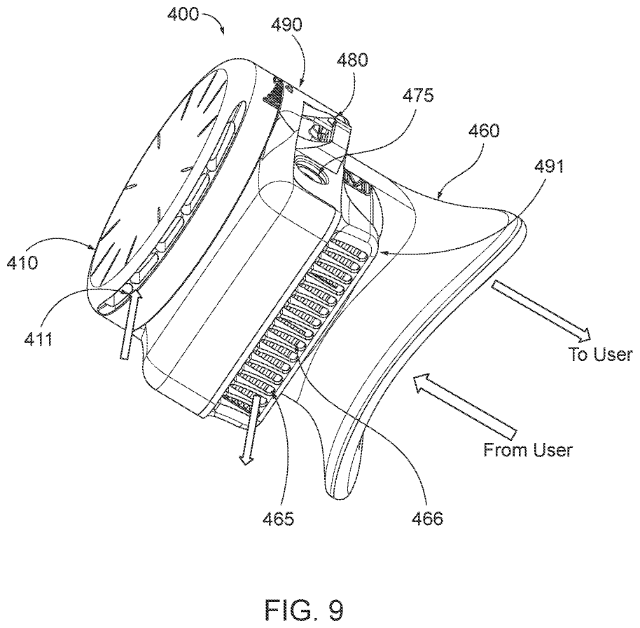

FIG. 9 shows a bottom perspective view of the assembled apparatus 400. As seen in this view, air enters the apparatus via air inlet holes 411, travels through the apparatus as shown in FIGS. 7 and 8 and to the user form mask 460. Exhaled air is returned to the mask 460 and after passing through filter 466 exits the apparatus via opening 464.

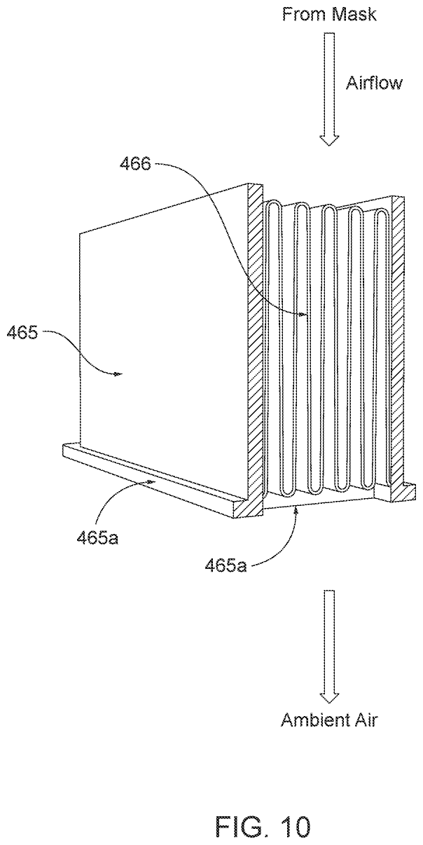

FIG. 10 shows a perspective cross-section schematic view of the exhaled air filter cartridge 465. The exhaled air filter cartridge provides for provides for decontaminating air exhaled from the user, thereby protecting others in the vicinity from viral particles the user may exhale. The cartridge 465 comprises a plastic body comprising box having four sides and open top and bottom ends. The exterior faces of the sides are configured to engage the interior surface of the chamber 463 of mask 460 (not shown). Flange 465a is configured to engage the perimeter of opening 464 of mask 460 (not shown). Disposed inside the filter cartridge 465 is a pleated air filter 466. Filter 466 is configured to catch droplets above 0.5 μm. The filter medium comprises for example, a copper mesh or is impregnated with copper particles or infused with copper, which can kill harmful bacteria and viruses in a very short time (less than a few minutes). The copper is capable of decontaminating the filter and air flowing through it.

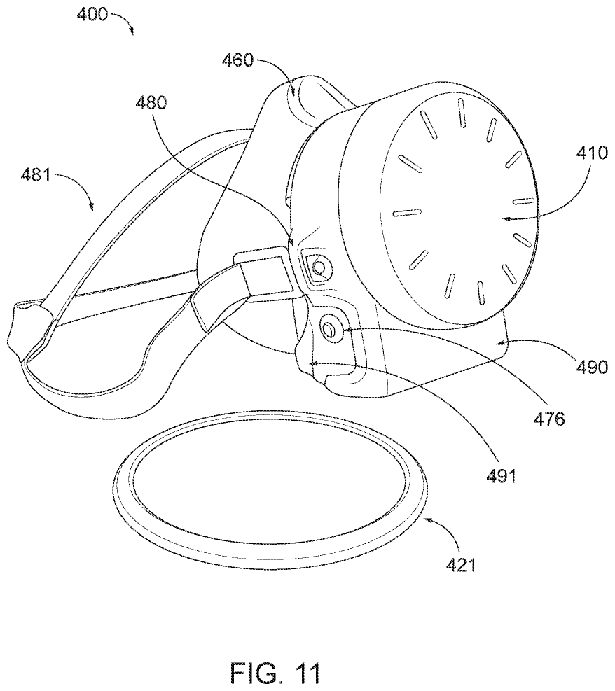

FIG. 11 depicts an embodiment of apparatus 400, in which a head harness 481 is attached to strap connector 480. Harness 481 comprises a first strap configured to wrap around the rear of a user's head and a second strap configured to wrap around the top of a user's head. A control button 476 is disposed in hole 475a in rear housing body 491. Also shown is a HEPA filter 421.

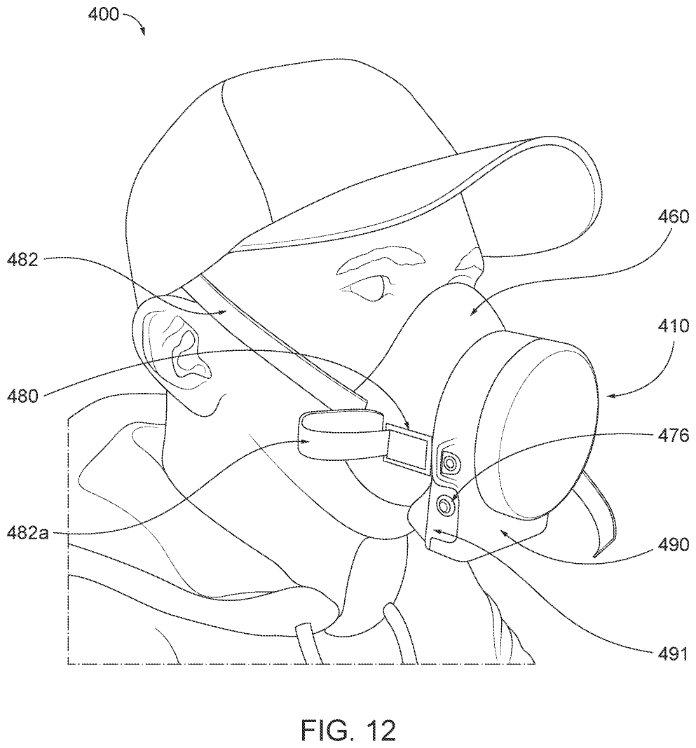

FIG. 12 depicts an embodiment of apparatus 400 worn by a user, in which a head strap 482 is attached to strap connector 480. Head strap 482 comprises a single strap configured to wrap around the back of the user's head. Strap 482 comprises ends 482a that can be pulled through strap connector 480 to adjust the strap length. The straps 481 or 482 or any other strap configuration may comprise an elastic fabric and/or elastomeric plastic to provide a snug fit on a user's head. Other optional strap length adjustment features may comprise, for example, buckles including frame-and-prong, plate, box-frame or O-Ring/D-Ring types, or ratchets that the strap passes through and is adjustably held in a desired length depending on desired fit for a given user.

Similar to that described above in relation to embodiment 1 in FIGS. 1 through 3, air movement through the apparatus 400 can be in response to a user's respiration alone. In some embodiments, air movement can be augmented and controlled by power supplied to impeller 440, controlled by the user to increase air flow in a BOOST configuration controlled by user input at fixed values such as low/medium/high, or variably controlled as described above.

In preferred embodiments, air movement through an air purification apparatus 500, including an apparatus physically configured as apparatus 400 in FIGS. 4 through 12, can be autonomously controlled by a computer controller comprising one or more processors in an “Auto” airflow control mode. In embodiments, a user can switch from “Auto” to “Manual” mode and adjust airflow speed as described above. In embodiments, the computer-instantiated components of air purification device 500 can be located on printed circuit board assembly 430 of air purification device 400.

During the human breath process, the air pressure inside the mask is increasing or decreasing, which affects the impeller rotation speed, which can be sensed by one or more sensors. According to impeller rotation speed, a processor in the controller averages read values and the system determines the dynamics of the impeller speed increase or decrease. Comparing the difference between increase and decrease with an empirically determined dimensionless factor, the processor uses an algorithm to gradually decrease or increase the power supplied to the impeller. In a parallel mode, the system is constantly analyzing blower power consumption, which is fine tuning and adjusting the power consumption using pulse width modulation.

As used herein, processor, microprocessor, and/or digital processor may include any type of digital processing device such as, without limitation, digital signal processors (“DSPs”), reduced instruction set computers (“RISC”), complex instruction set computers (“CISC”) processors, microprocessors, gate arrays (e.g., field programmable gate arrays (“FPGAs”)), programmable logic device (“PLDs”), reconfigurable computer fabrics (“RCFs”), array processors, secure microprocessors, and application-specific integrated circuits (“ASICs”). Such digital processors may be contained on a single unitary integrated circuit die or distributed across multiple components.

As used herein, computer program and/or software may include any sequence or human or machine cognizable steps which perform a function. Such computer program and/or software may be rendered in any programming language or environment including, for example, C/C++, C#, Fortran, COBOL, MATLAB™, PASCAL, GO, RUST, SCALA, Python, assembly language, markup languages (e.g., HTML, SGML, XML, VoXML), and the like, as well as object-oriented environments such as the Common Object Request Broker Architecture (“CORBA”), JAVA™ (including J2ME, Java Beans, etc.), Binary Runtime Environment (e.g., “BREW”), and the like.

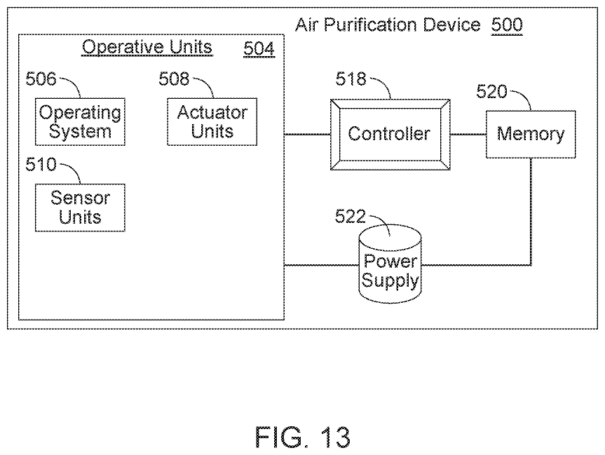

FIG. 13 is a functional block diagram of an air purification device in accordance with some principles of this disclosure. As illustrated in FIG. 13, air purification device 500 may include controller 518, memory 520, actuator units 108 and sensor units 510, as well as other components and subcomponents (e.g., some of which may not be illustrated). Although a specific embodiment is illustrated in FIG. 13, it is appreciated that the architecture may be varied in certain embodiments as would be readily apparent to one of ordinary skill given the contents of the present disclosure.

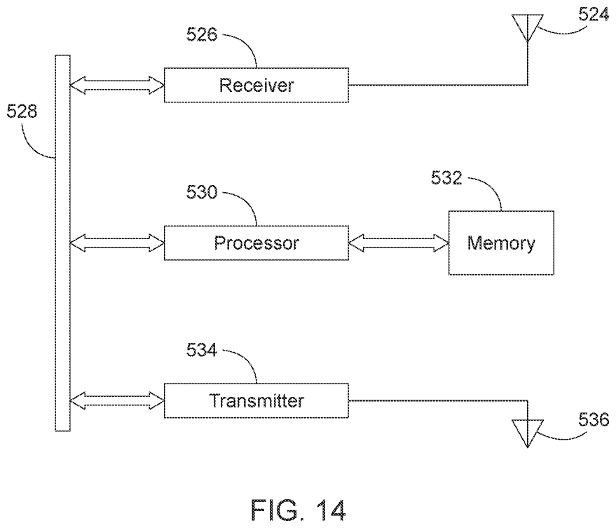

The controller 518 may control the various operations performed by the air purification device. The controller may include and/or comprise one or more processors (e.g., microprocessors) and other peripherals as shown in FIG. 14. As used herein, processor, processing device, microprocessor, and/or digital processor may include any type of digital processor such as, without limitation, digital signal processors (“DSPs”), reduced instruction set computers (“RISC”), complex instruction set computers (“CISC”), microprocessors, gate arrays (e.g., field programmable gate arrays (“FPGAs”)), programmable logic device (“PLDs”), reconfigurable computer fabrics (“RCFs”), array processors, secure microprocessors and application-specific integrated circuits (“ASICs”).

The controller 518 may be operatively and/or communicatively coupled to memory 520. Memory 520 may include any type of integrated circuit or other storage device configured to store digital data including, without limitation, read-only memory (“ROM”), random access memory (“RAM”), non-volatile random access memory (“NVRAM”), programmable read-only memory (“PROM”), electrically erasable programmable read-only memory (“EEPROM”), dynamic random-access memory (“DRAM”), Mobile DRAM, synchronous DRAM (“SDRAM”), double data rate SDRAM (“DDR/2 SDRAM”), extended data output (“EDO”) RAM, fast page mode RAM (“FPM”), reduced latency DRAM (“RLDRAM”), static RAM (“SRAM”), flash memory (e.g., NAND/NOR), memristor memory, pseudostatic RAM (“PSRAM”), etc. Memory may provide computer-readable instructions and data to controller 518. For example, memory 520 may be a non-transitory, computer-readable storage apparatus and/or medium having a plurality of instructions stored thereon, the instructions being executable by a processing apparatus (e.g., controller 518) to operate air purification device 500. In some cases, the computer-readable instructions may be configured to, when executed by the processing apparatus, cause the processing apparatus to perform the various methods, features, and/or functionality described in this disclosure. Accordingly, controller 518 may perform logical and/or arithmetic operations based on program instructions stored within memory. In some exemplary embodiments, memory may store a library of sensor data obtained from the one or more pressure sensors.

Operative units 504 may be coupled to controller 518, or any other controller, to perform the various operations described in this disclosure. Throughout this disclosure, reference may be to various controllers and/or processors. In some embodiments, a single controller may serve as the various controllers and/or processors described. In other embodiments different controllers and/or processors may be used, such as controllers and/or processors used particularly for one or more operative units 104. The controller 518 may send and/or receive signals, such as power signals, status signals, data signals, electrical signals, and/or any other desirable signals, including discrete and analog signals to operative units. The controller 518 may coordinate and/or manage operative units, and/or set timings (e.g., synchronously or asynchronously), turn off/on control power budgets, send interrogatory signals, receive and/or send statuses, and/or perform any operations for running features of the air purification device.

Operative units 504 may include various units that perform functions for the air purification device. For example, operative units 504 includes at least actuator unit(s) 508 and sensor units 510. Operative units may also comprise other units such as specifically configured task units (not shown) that provide the various functionality of air purification device. In exemplary embodiments, operative units may be instantiated in software, hardware, or both software and hardware. For example, in some cases, units of operative units 104 may comprise computer implemented instructions executed by a controller. In exemplary embodiments, units of operative unit 104 may comprise hardcoded logic (e.g., ASICS). In exemplary embodiments, units of operative units may comprise both computer-implemented instructions executed by a controller and hardcoded logic. Where operative units are implemented in part in software, operative units may include units/modules of code configured to provide one or more functionalities.

In exemplary embodiments, operating system 506 may be configured to manage memory 520, controller 518, power supply 522, modules in operative units 504, and/or any software, hardware, and/or features of the air purification device. For example, and without limitation, operating system 510 may include device drivers to manage hardware recourses for the device.

Actuator units 508 may include actuators such as electric motors and/or any way of driving an actuator known in the art. In particular, activator units may include micromotor(s) for driving impeller 440 rotationally to move air through the device.

According to exemplary embodiments, sensor units 510 may comprise systems and/or methods that may detect characteristics within and/or around air purification device. Sensor units 510 may comprise a plurality and/or a combination of sensors. Sensor units 510 may include sensors that are internal or external to the air purification device. In some cases, sensor units 510 may include one or more exteroceptive sensors, such as an ambient pressure sensor 57 for measuring atmospheric pressure outside the air purification device. According to some exemplary embodiments, sensor units 510 may collect raw measurements (e.g., currents, voltages, resistances, gate logic, etc.) and/or transformed measurements. In some cases, measurements may be aggregated and/or summarized. Sensor units 510 may include sensors that may measure internal characteristics of the air purification device. For example, sensor units 510 may measure temperature, power levels, statuses, and/or any characteristic of air purification device. In particular sensor units 510 may measure pressure internal to the air purification device at one or more locations, including the filter section 420, UV treatment section 450 and/or mask 460. Such data may be stored in data structures, such as matrices, arrays, queues, lists, arrays, stacks, bags, etc.

According to exemplary embodiments, user interface units may be configured to enable a user to interact with the air purification device. For example, user interface units may include for example touch panels, buttons, sliders, dials, keypads/keyboards, and the like to enable a user to provide input to the device. User interface units may also comprise units to provide information to a user, including for example displays, such as, without limitation, liquid crystal display (“LCDs”), light-emitting diode (“LED”) displays, organic LED displays, indicator lights, and/or devices known in the art for visual presentation.

Power supply 522 comprises any power supply as previously described including a battery or power cord connected to an external power source.

As used herein, the controller of the air purification device comprises a controller executing computer readable instructions stored on a non-transitory computer readable storage apparatus, such as memory, as would be appreciated by one skilled in the art.

As shown in FIG. 14, the architecture of a controller 518 for the air purification device includes a data bus 528, a receiver 526, a transmitter 534, at least one processor 530, and a memory 532. As one skilled in the art would appreciate, the data bus 528 is the means of communication between the different components—receiver 526, processor 530, and transmitter 534—in the processing device. The receiver 526, the processor 530 and the transmitter 534 all communicate with each other via the data bus 528. The processor 530 is configurable to access the memory 532, which stores computer code or computer readable instructions in order for the processor to execute specialized algorithms for operating the device. Memory 532 may be the same as or different from memory 520. The receiver 526 is configurable to receive input signals 524 that may comprise signals from a plurality of operative units 504 including, but not limited to, sensor data from sensor units 510, user inputs from user interface units, motor feedback, external communication signals (e.g., from a remote server), and/or any other signal from an operative unit 504 requiring further processing. The receiver 526 communicates these received signals 524 to the processor 530 via the data bus 528. The processor 530 executes the algorithms, as discussed below, by accessing specialized computer-readable instructions from the memory. The processor 530 may communicate output signals to the transmitter 534 via data bus 528. The transmitter 534 may be configurable to further communicate the output signals 536 to a plurality of operative units 504.

One of ordinary skill in the art would appreciate that a controller 518 of the air purification device may include one or more processors 530 and may further include other peripheral devices used for processing information, such as ASICS, DPS, proportional-integral-derivative (“PID”) controllers, and/or other peripherals (e.g., analog to digital converters). In some instances, peripheral devices are used as a means for intercommunication between the controller and operative units (e.g., digital to analog converters and/or amplifiers for producing actuator signals). Accordingly, as used herein, the controller executing computer readable instructions to perform a function may include one or more processors thereof executing computer readable instructions and, in some instances, the use of any hardware peripherals known within the art. Controller 518 may comprise various processors 530 and peripherals integrated into a single circuit die or distributed to various locations of the air purification device which receive, process, and output information to/from operative unit 504 locations of the air purification device to effectuate control of locations of the air purification device in accordance with instructions stored in a memory. For example, the controller may include a plurality of processors 530 for performing high level tasks (e.g., planning an air flow protocol within the device) and processors 530 for performing low-level tasks (e.g., producing actuator signals in accordance with the air flow protocol, or producing output signals to the user interface based on sensor inputs).

As indicated above, air movement through the apparatus 500 can be autonomously controlled by a computer controller in an “Auto” airflow control mode.

Accordingly, the air purification device comprises an air inlet and an air outlet; an air passage in fluid communication with the air inlet and the air outlet; an impeller in the internal air passage; a sensor to measure rotational speed of the impeller; a computer instantiated controller; and a non-transitory computer readable storage medium comprising a plurality of computer readable instructions embodied thereon which, when executed by the controller, causes the controller to: receive a plurality of measurements from the sensor, the measurements providing information about impeller rotational speed in the air passage during respiration of a user of the air purification device; average the values obtained from a plurality of measurements obtained during user inhalations and average the values obtained from a plurality of measurements obtained during user exhalations; determine the difference between the average rotational speed value obtained during inhalations and the average rotational speed value obtained during exhalations; compare the difference between the average rotational speed value obtained during inhalations and the average rotational speed value obtained during exhalations with an empirically determined dimensionless factor; and provide instructions to an actuator coupled to the impeller to decrease or increase power supplied to the impeller to decrease or increase impeller rotation speed.

When a user puts on the apparatus 500, such as embodied in air purification device 400, and powers it up in Auto control mode, an actuator comprising a micro-motor begins turning the impeller 440 at an initial rotational speed R₀, drawing air into the device via air inlet holes 411. As the user respires, air is pulled through the apparatus during inhalations, reducing the dynamic pressure in the air passage of the device, causing the impeller to rotate at a faster speed R_{I}. During exhalations, air would be pushed into the device via opening 461, increasing the dynamic pressure in the air passage of the device, causing the impeller to rotate at a slower speed R_{E}. Thus, dynamic air pressure inside the device and impeller rotational speeds follow a generally sinusoidal wave function as the user breathes through the device.

A sensor associated with the impeller 440 measures the dynamic rotational speed of the impeller and sends the measurements to the controller 518. A processor on controller 518 follows computer readable instructions to separately average the impeller speeds R_{I} and R_{E} to obtain aveR_{I} and aveR_{E}. The processor then compares the difference between aveR_{I} and aveR_{E} and the initial speed R₀ to determine the average increase or decrease in rotational speed and, using an algorithm, converts the differences in rotational speed to define a dimensionless user-dependent respiration use factor. The algorithm is used for all users to detect and analyze breath patterns and mask behavior for each individual user to provide a customized respiration use factor for each user.

The processor compares the user-dependent respiration factor to an empirically determined dimensionless factor determined at the time of manufacture and stored in memory 520 or 532 in controller 518. The parameters for the empirically determined factor are selected in a way to respond to an average user's average air consumption rate.

Based on the comparison between the user-dependent user factor to the empirically determined factor, the controller 518 provides instructions to the actuator coupled to the impeller to decrease or increase power supplied to the impeller to decrease or increase impeller rotation speed. Impeller rotational speed is always adjusted to follow average air demand and provide average airflow (volume) for the individual user.

For example, the impeller speed may be increased during a user's exhalation so that the internal air pressure inside the device is greater than that inside mask 460, so that exhaled air does not enter the device via opening 461 and instead passes through exhalation valve 462 toward exhaled air filter 465.

One can appreciate that a user's respiration is not constant over time and may vary in intensity, periodicity, duration, etc., influenced by a user's activity, emotional state, etc. For example, a user breathes harder when the user's exertion increases, or a user may hold one's breathe while concentrating.

As the user continues to breathe through the device 400, the controller continually receives impeller rotational speed measurements and determines moving averages related to aveR_{I} and aveR_{E} to continually define the user-dependent factor for comparison to the empirically determined factor and continually adjust impeller speed based on the comparison.

In embodiments, the air purification device further comprises a power consumption sensor coupled to the actuator of the impeller, wherein the controller is configured to:

receive a plurality of measurements from the power consumption sensor, the measurements providing information about power consumption during respiration of a user of the air purification device; analyze the power consumption information provided by the plurality of measurements; compare impeller rotational speed to power average values using pulse width modulation (PWM); modify the user-dependent respiration use factor to optimize power consumption; and provide instructions to the actuator coupled to the impeller to decrease or increase power supplied to the impeller to decrease or increase impeller rotation speed in discrete segments.

As discussed above, the user's respiration is generally sinusoidal, and the power supplied to the impeller in response may also be generally sinusoidal. Power consumption by the impeller can be optimized, that is, made more efficient, by pulse width modulation.

Pulse width modulation (PWM), or pulse-duration modulation (PDM), is a method of reducing the average power delivered by an electrical signal, by effectively chopping it up into discrete parts. The average value of voltage (and current) fed to the load is controlled by turning the switch between supply and load on and off at a fast rate. The longer the switch is on compared to the off periods, the higher the total power supplied to the load. PWM is particularly suited for running inertial loads such as the impeller motor, which is not easily affected by this discrete switching, because of its rotation. The PWM switching frequency has to be high enough not to affect the load, which is to say that the resultant waveform perceived by the load must be as smooth as possible.

The PWM switching frequency may be between a few kilohertz (kHz) and tens of kHz for the motor drive or even higher; such as into the tens or hundreds of kHz. A major advantage of PWM is that power loss in the switching devices is very low. When a switch is off there is practically no current, and when it is on and power is being transferred to the load, there is almost no voltage drop across the switch. Power loss, being the product of voltage and current, is thus in both cases close to zero. PWM also works well with digital controls, which, because of their on/off nature, can easily set the needed duty cycle.

In embodiments, the controller in the device comprises an integrated PWM microcontroller using internal programming interfaces to effect direct current (DC) motor control to the impeller actuator.

These analytics are recurring in a time period which we call reaction time, which may be less than 10 seconds, such as 3 to 5 seconds for each cycle time. The system is making decisions to increase or decrease average airflow by changing impeller speed in response to the user's respiration, which will be analyzed again from the beginning of this cycle to provide a moving average of respiration and power consumption for each reaction time cycle.

As indicated above, a user may switch from Auto control mode to Manual control mode and set a user-desired impeller speed using a user interface. For example, a user may select a faster impeller speed if a period of greater user activity is anticipated. In embodiments, the user selected setting may provide a constant impeller speed. In other embodiments, the controller may determine the timing of a user's respiration cycle and apply a sinusoidal wave variation of impeller speed to the user-selected speed setting. In other embodiments, the user selects a desired initial impeller speed and the controller applies the methodology described above to the user-selected speed setting to customize the impeller speed to the user's respiration.

Also provided is a non-transitory computer readable storage medium comprising a plurality of computer readable instructions embodied thereon which, when executed by a controller of the air purification device 500 causes the controller to: receive a plurality of measurements from the sensor, the measurements providing information about impeller rotational speed in the air passage during respiration of a user of the air purification device; average the values obtained from a plurality of measurements obtained during user inhalations and average the values obtained from a plurality of measurements obtained during user exhalations; determine the difference between the average rotational speed value obtained during inhalations and the average rotational speed value obtained during exhalations; compare the difference between the average rotational speed value obtained during inhalations and the average rotational speed value obtained during exhalations with an empirically determined dimensionless factor; and provide instructions to an actuator coupled to the impeller to decrease or increase power supplied to the impeller to decrease or increase impeller rotation speed.

In embodiments wherein the air purification device further comprises a power consumption sensor coupled to the actuator of the impeller, the non-transitory computer readable storage medium further cause the controller to: receive a plurality of measurements from the power consumption sensor, the measurements providing information about power consumption during respiration of a user of the air purification device; analyze the information provided by the plurality of measurements; and modify the abovementioned algorithm.

Also provided is a method for controlling air flow through the air purification device comprising an air inlet and an air outlet; an air passage in fluid communication with the air inlet and the air outlet; an impeller in the internal air passage; and a sensor to measure rotational speed of the impeller; the method comprising the controller: receiving a plurality of measurements from the sensor, the measurements providing information about impeller rotational speed in the air passage during respiration of a user of the air purification device; averaging the values obtained from a plurality of measurements obtained during user inhalations and average the values obtained from a plurality of measurements obtained during user exhalations; determining the difference between the average rotational speed value obtained during inhalations and the average rotational speed value obtained during exhalations; comparing the difference between the average rotational speed value obtained during inhalations and the average rotational speed value obtained during exhalations with an empirically determined dimensionless factor; and providing instructions to an actuator coupled to the impeller to decrease or increase power supplied to the impeller to decrease or increase impeller rotation speed.

In embodiments wherein the air purification device further comprises a power consumption sensor coupled to the actuator of the impeller, the method further comprises the controller: receiving a plurality of measurements from the power consumption sensor, the measurements providing information about power consumption during respiration of a user of the air purification device; analyzing the power consumption information provided by the plurality of measurements; comparing impeller rotational speed to power average values using pulse width modulation (PWM); modifying the user-dependent respiration use factor to optimize power consumption; and providing instructions to the actuator coupled to the impeller to decrease or increase power supplied to the impeller to decrease or increase impeller rotation speed in discrete segments.

In some embodiments, the controller may comprise machine learning algorithms to receive and process sensor inputs 524 to learn, for example, rotational speeds of the impeller due to respiration patterns of a user and impeller power consumption and provide output signals 536 to actuators 508 to control air flow in the device, such as for controlling impeller speed. In some embodiments, a neural network is employed to receive and process sensor inputs 524 to learn patterns and adjust weighting factors in the algorithm used to provide signal outputs to control air flow in the device.

In some embodiments, the apparatus 400 may further comprise a negative ion generator as described above for apparatus 1.

As shown in FIGS. 1 through 3, the air purification apparatus is depicted as a unitary device wherein the filter section, air mover, and UV treatment section are configured generally in line with and directly in front of the user's mouth and nose, but this configuration is not limiting.

In some exemplary embodiments, such as shown in FIGS. 4 through 12, a unitary air purification apparatus may be oriented so that the air inlet is at the bottom of the mask as worn by the user and the air outlet is at the upper portion of the oronasal area of the mask.

In other exemplary embodiments, a unitary air purification apparatus is configured to the side of the mask, such as where the air outlet faces the cheek and lower jaw area of the user and the air inlet faces obliquely away from the user. In still other exemplary embodiments, the disclosed air purification apparatus is oriented across the face of the mask near the mouth and chin area, wherein the air inlet faces toward the side of the user and the air outlet engages the mask near the nose of the user.

In still other exemplary embodiments, the air purification apparatus may be configured to be remote from the mask and the air outlet is connected to the inlet of the mask via a hose or tubing. In such embodiments, the air purification apparatus may be instantiated in a chest pack, backpack, waist pack or fanny pack.

As discussed above and shown in the Figures, the air purification apparatus may be a unitary device with all sections arranged in linear configuration. Alternatively, the air purification apparatus may comprise two or more submodules that each contain one, some, but not all of the components described herein, wherein the submodules are in fluid communication via an extended air passage. For example but not limitation, a first submodule may comprise the filter section, a second module may comprise the UV treatment section and a third module may comprise an air movement assist module.

Any of the preceding alternative embodiments may, for example, provide improved performance, weight distribution, user comfort and/or field of view.

In some embodiments, the disclosed apparatus may comprise more than one of the individual components. For example, the disclosed apparatus may comprise two modules comprising the air inlet and filter sections in fluid communication with a third module comprising an air movement component and the UV treatment section.

In the exemplary embodiments shown in FIGS. 1 through 12, the mask is an oronasal mask, but this is not limiting. In other embodiments, the mask may further comprise an integrated transparent eye protection component such as goggles, a viewing plate in front of the eye region of the user, an extended viewing plate that is transparent over a greater portion of the user's face such as revealing the user's nose, or the user's nose and mouth, to provide a full face mask. Similarly the PPE may be a hood or shield that covers the user's head. One of skill in the art can appreciate that features such as the seal areas, harness or straps to secure the PPE to the user, etc. may be specific to the type of mask, hood or shield envisioned, but such features do not affect the essential features of the disclosed air purification apparatus.

Although the disclosed apparatus can be integrated with an article of PPE such as a mask to provide a complete unit, the disclosed apparatus may alternatively be configured with fittings at the air inlet, air outlet or both, to allow attachment of the apparatus to an existing article of PPE such as a mask, hood shield, or a medical treatment device.

The foregoing discussion relates to use of the disclosed apparatus to provide purified air for a user to breathe, wherein the user is presumed to be uninfected.

However, in other aspects the apparatus is used to purify exhaled air from an infected user or patient. These aspects may include the following embodiments.

The apparatus is configured to provide air purification in a closed environment such as an emergency room, treatment room, patient room or intensive care unit to purify air that may be distributed in the closed environment to reduce the bioload of virus in the air. One can appreciate that the throughput of such room purifiers may be significantly greater than that needed for treatment of air for a single user such as described above.

In other embodiments, the air inlet comprises a fitting to attach the apparatus to an article of personal protective equipment, such as a respirator mask, hood or shield, or a medical treatment device. In such embodiments, the air inlet may be in fluid communication with an exhalation valve of the personal protective equipment or the medical treatment device.

The air inlet is integrated into an article of personal protective equipment, such as a respirator mask, hood or shield, or a medical treatment device. In such embodiments, the air inlet may be in fluid communication with an exhalation valve of the personal protective equipment or the medical treatment device.

In the above embodiments, the mask, hood or shield provides clean breathing air to the patient but the breath exhaled from the infected patient contains virus particles. Venting the exhalations into the environment around the patient contributes to dispersion of virus particles to other individuals. Therefore, it is desirable to treat exhalations from the patient to kill virus particles. Use of the disclosed apparatus accomplishes this when it is configured to receive exhalations from an infected individual into the air inlet of the apparatus.

In some embodiments, the patient can breathe unassisted and the exhalations are driven out of the exhalation valve of a mask into the apparatus by the normal respiration of the patient. For patent comfort it may be desirable to provide a breathing space that is not confined to a close-fitting mask, such as a shield or tent. In other situations such as during intubation and extubation procedures, the patient cannot wear a mask and significant aerosols including virus particles are ejected into the air around the patient, and a shield hood or tent is useful to contain the aerosols in a relatively confined space to be collected and purified by the disclosed apparatus. In some exemplary embodiments, negative air pressure such as from a vacuum pump or aspirator can be applied to the air outlet of the apparatus to pull air through the apparatus.

In exemplary embodiments the air purification apparatus may be desirably positioned away from a patient's mask, hood or shield and air brought to the air inlet via a hose or tube. In such embodiments the air purification apparatus may be placed on a bedside table or equipment cart or hung from a bedrail or IV pole.

In still other embodiments the air purification apparatus is used in conjunction with a ventilator. A ventilator is used to provide powered respiration to patient who are not capable of breathing on their own, or even with a positive pressure assist. The disclosed apparatus can purify air expelled from the lungs of the patient during ventilation.

In some instances, such as when a health care worker is infected with the virus but is presymptomatic or asymptomatic and needs to continue treating patients, it may be desirable to provide purified air for the user to breathe and purify the user's exhaled breath. In such instances, the outlet of a first disclosed apparatus is in fluid communication with the inlet of the user's mask and the inlet of a second disclosed apparatus is in fluid communication with the outlet of the user's mask. It can be appreciated that one-way valves may be required to manage airflow so that air passes into the air inlet of the first disclosed apparatus, through the first apparatus and out its outlet into the mask and from the mask into the inlet of the second disclosed apparatus for purification.

Embodiments and aspects of the invention include those wherein:

The air-purification apparatus comprises an air inlet and an air outlet; an air passage in fluid communication with the air inlet and the air outlet comprising a filtration section comprising a HEPA filter; an ultraviolet (UV) light treatment section; and optionally, an ionization section.

The air inlet comprises a filter cap.

The filtration section further comprises one or more pre-filters.

The filtration section comprises two types of pre-filters selected from a hydrophobic layer and a fabric mesh layer.

The HEPA filter is treated with UV light while air passes through it.

The inner surface of the air passage in the UV light treatment section is coated with silver ions or polished aluminum.

The air passage in the UV light treatment section comprises a spiral pathway.

The air passage in the UV light treatment section comprises at least two bends comprising angles of at least 90 degrees wherein the UV light treats the air in the portion of the air passage between the at least two bends.

The air passage in the UV light treatment section comprises UV-C emitting diodes at the at least two bends.

The air passage in the UV light treatment section comprises a Z-shaped passage.

The air passage in the UV light treatment section comprises a labyrinthine pathway.

The air passage in the UV light treatment section is configured to provide a residence time of air sufficient to kill virus particles contained therein.

The UV light is UV-C light with wavelengths in a range of 254 nm to 300 nm.

The ionization section generates negative ions.

The apparatus further comprises a propeller or impeller to move air through the air passage.

The apparatus further comprises a power supply.

The apparatus wherein the power supply comprises a battery.

The apparatus optionally further comprises an external UV light.

In some aspects the apparatus is used to purify breathing air prior to its inhalation to protect an uninfected user. These aspects may include the following embodiments.

The air outlet comprises a fitting to attach the apparatus to an article of personal protective equipment, such as a respirator mask, hood or shield, or a medical treatment device.

The air outlet is integrated into an article of personal protective equipment, such as a respirator mask, hood or shield.

The apparatus wherein the respirator mask, hood or shield further comprises an exhalation valve.

In some aspects the apparatus is used to purify exhaled air from an infected user or patient. These aspects may include the following embodiments.

The air inlet comprises a fitting to attach the apparatus to an article of personal protective equipment, such as a respirator mask, hood or shield, or a medical treatment device. In such embodiments, the air inlet may be in fluid communication with an exhalation valve of the personal protective equipment or the medical treatment device.

The air inlet is integrated into an article of personal protective equipment, such as a respirator mask, hood or shield, or a medical treatment device. In such embodiments, the air inlet may be in fluid communication with an exhalation valve of the personal protective equipment or the medical treatment device.

One can appreciate from the preceding discussion that the air purification apparatus can be configured in a wide variety of configurations, allowing significant design possibilities, none of which are limiting.

While the disclosure has been illustrated and described in detail in the drawings and foregoing description, such illustration and description are to be considered illustrative or exemplary and not restrictive. The disclosure is not limited to the disclosed embodiments. Variations to the disclosed embodiments and/or implementations may be understood and effected by those skilled in the art in practicing the claimed disclosure, from a study of the drawings, the disclosure and the appended claims.

It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term “including” should be read to mean “including, without limitation,” “including but not limited to,” or the like; the term “comprising” as used herein is synonymous with “including,” “containing,” or “characterized by,” and is inclusive or open-ended and does not exclude additional, unrecited elements or method steps; the term “having” should be interpreted as “having at least;” the term “such as” should be interpreted as “such as, without limitation;” the term ‘includes” should be interpreted as “includes but is not limited to;” the term “example” or the abbreviation “e.g.” is used to provide exemplary instances of the item in discussion, not an exhaustive or limiting list thereof, and should be interpreted as “example, but without limitation;” the term “illustration” is used to provide illustrative instances of the item in discussion, not an exhaustive or limiting list thereof, and should be interpreted as “illustration, but without limitation.” Adjectives such as “known,” “normal,” “standard,” and terms of similar meaning should not be construed as limiting the item described to a given time period or to an item available as of a given time, but instead should be read to encompass known, normal, or standard technologies that may be available or known now or at any time in the future; and use of terms like “preferably,” “preferred,” “desired,” or “desirable,” and words of similar meaning should not be understood as implying that certain features are critical, essential, or even important to the structure or function of the present disclosure, but instead as merely intended to highlight alternative or additional features that may or may not be utilized in a particular embodiment. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should be read as “and/or” unless expressly stated otherwise. The terms “about” or “approximate” and the like are synonymous and are used to indicate that the value modified by the term has an understood range associated with it, where the range may be ±20%, ±15%, ±10%, ±5%, or ±1%.

1. An air-purification apparatus comprising
an air inlet and an air outlet;
an air passage in fluid communication with the air inlet and the air outlet comprising
a filtration section comprising a HEPA filter wherein the HEPA filter is treated with UV light while air passes through it; and
an ultraviolet (UV) light treatment section wherein filtered air from the filtration section is treated with UV light.

2. The air-purification apparatus of claim 1 wherein the air inlet comprises a filter cap comprising a plurality of air inlet holes on the lower portion of the rim of the filter cap.

3. The air-purification apparatus of claim 1 wherein the inner surface of the filter cap comprises polished aluminum to reflect UV light toward the front face of the HEPA filter.

4. The air-purification apparatus of claim 3 wherein the HEPA filter further comprises a hydrophobic layer.

5. The air-purification apparatus of claim 1 wherein the inner surface of the air passage in the UV light treatment section is coated with silver ions or polished aluminum.

6. The air-purification apparatus of claim 1 wherein the air passage in the UV light treatment section comprises a spiral pathway.

7. The air-purification apparatus of claim 1 wherein the air passage comprises at least two bends comprising angles of at least 90 degrees wherein the UV light treats the air in the portion of the air passage between the at least two bends.

8. The air-purification apparatus of claim 1 wherein the UV light is UV-C light with wavelengths in a range of 254 nm to 300 nm.

9. The air-purification apparatus of claim 1 further comprises an exhalation valve.

10. The air-purification apparatus of claim 1 further comprises a filter to filter and treat exhaled air.

11. The air-purification apparatus of claim 1 wherein the apparatus further comprises an impeller disposed in the UV filter section to move air through the air passage.

12. The air-purification apparatus of claim 1 wherein the air outlet comprises a fitting to attach the apparatus to an article of personal protective equipment or a medical treatment device.

13. The air-purification apparatus of claim 12 wherein the article of personal protective equipment comprises a respirator mask, hood or shield.

14. The air-purification apparatus of claim 1 wherein the apparatus further comprises a controller configured to autonomously control airflow through the apparatus by adjusting impeller rotational speed.

15. An air purification device, comprising
an air inlet and an air outlet;
an internal air passage in fluid communication with the air inlet and the air outlet;
an impeller in the internal air passage;
a sensor to measure rotational speed of the impeller;
a computer instantiated controller; and
a non-transitory computer readable storage medium comprising a plurality of computer readable instructions embodied thereon which, when executed by the controller, causes the controller to:
receive a plurality of measurements from the sensor, the measurements providing information about impeller rotational speed in the air passage during respiration of a user of the air purification device;
average the values obtained from a plurality of measurements obtained during user inhalations and average the values obtained from a plurality of measurements obtained during user exhalations;
determine the difference between the average rotational speed value obtained during inhalations and the average rotational speed value obtained during exhalations;
compare the difference between the average rotational speed value obtained during inhalations and the average rotational speed value obtained during exhalations with an empirically determined dimensionless factor; and
provide instructions to an actuator coupled to the impeller to decrease or increase power supplied to the impeller to decrease or increase impeller rotation speed.

16. The air purification device of claim 15 further comprising a power consumption sensor coupled to the actuator of the impeller, wherein the controller is configured to:
receive a plurality of measurements from the power consumption sensor, the measurements providing information about power consumption during respiration of a user of the air purification device;
analyze the power consumption information provided by the plurality of measurements;
compare impeller rotational speed to power average values using pulse width modulation (PWM);
modify the user-dependent respiration use factor to optimize power consumption; and
provide instructions to the actuator coupled to the impeller to decrease or increase power supplied to the impeller to decrease or increase impeller rotation speed in discrete segments.

17. A non-transitory computer readable storage medium comprising a plurality of computer readable instructions embodied thereon which, when executed by a controller of an air purification device comprising an air inlet and an air outlet; an internal air passage in fluid communication with the air inlet and the air outlet; an impeller in the internal air passage; and a sensor to measure rotational speed of the impeller;
wherein the plurality of computer readable instructions causes the controller to:
receive a plurality of measurements from the sensor, the measurements providing information about impeller rotational speed in the air passage during respiration of a user of the air purification device;
average the values obtained from a plurality of measurements obtained during user inhalations and average the values obtained from a plurality of measurements obtained during user exhalations;
determine the difference between the average rotational speed value obtained during inhalations and the average rotational speed value obtained during exhalations;
compare the difference between the average rotational speed value obtained during inhalations and the average rotational speed value obtained during exhalations with an empirically determined dimensionless factor; and
provide instructions to an actuator coupled to the impeller to decrease or increase power supplied to the impeller to decrease or increase impeller rotation speed.

18. The non-transitory computer readable storage medium of claim 17 wherein the air purification device further comprises a power consumption sensor coupled to the actuator of the impeller, wherein the plurality of computer readable instructions further cause the controller to:
receive a plurality of measurements from the power consumption sensor, the measurements providing information about power consumption during respiration of a user of the air purification device;
analyze the power consumption information provided by the plurality of measurements;
compare impeller rotational speed to power average values using pulse width modulation (PWM);
modify the user-dependent respiration use factor to optimize power consumption; and
provide instructions to the actuator coupled to the impeller to decrease or increase power supplied to the impeller to decrease or increase impeller rotation speed in discrete segments.

19. A method for controlling air flow through an air purification device comprising an air inlet and an air outlet; an air passage in fluid communication with the air inlet and the air outlet; an impeller in the internal air passage; and a sensor to measure rotational speed of the impeller; the method comprising the controller:
receiving a plurality of measurements from the sensor, the measurements providing information about impeller rotational speed in the air passage during respiration of a user of the air purification device;
averaging the values obtained from a plurality of measurements obtained during user inhalations and average the values obtained from a plurality of measurements obtained during user exhalations;
determining the difference between the average rotational speed value obtained during inhalations and the average rotational speed value obtained during exhalations;
compare the difference between the average rotational speed value obtained during inhalations and the average rotational speed value obtained during exhalations with an empirically determined dimensionless factor; and
providing instructions to an actuator coupled to the impeller to decrease or increase power supplied to the impeller to decrease or increase impeller rotation speed.

20. The method of claim 19 wherein the air purification device further comprises a power consumption sensor coupled to the actuator of the impeller, wherein the method further comprises the controller:
receiving a plurality of measurements from the power consumption sensor, the measurements providing information about power consumption during respiration of a user of the air purification device;
analyzing the power consumption information provided by the plurality of measurements;
comparing impeller rotational speed to power average values using pulse width modulation (PWM);
modifying the user-dependent respiration use factor to optimize power consumption; and
providing instructions to the actuator coupled to the impeller to decrease or increase power supplied to the impeller to decrease or increase impeller rotation speed in discrete segments.
